(19) United States
(12) Patent Application Publication
(10) Pub. No.: US 2006/0178720 A1
(43) Pub. Date: Aug. 10, 2006

(54) Method and apparatus for sealing a lumen in an electrode assembly

(76) Inventors: Fysh Dadd, New South Wales (AU); Peter Gibson, New South Wales (AU); Martin Svehla, New South Wales (AU); Claudiu Treaba, New South Wales (AU)

Correspondence Address: JAGTIANI + GUTTAG, 10363-A DEMOCRACY LANE, FAIRFAX, VA, 22030, US

(21) Appl. No.: 10/539,764

(22) PCT Filed: Dec. 18, 2003
(86) PCT No.: PCT/AU03/01687

§ 371 Date: Mar. 20, 2006

(30) Foreign Application Priority Data
Dec. 19, 2002 (AU) 2002953473
Dec. 19, 2002 (AU) 2002953471
Dec. 19, 2002 (AU) 2002953469
Dec. 19, 2002 (GB) 2002953468

(51) Int. Cl. A61N 1/05

(52) U.S. Cl. 607/137

(57) ABSTRACT

An implantable tissue-stimulating device comprising a resiliently flexible elongate member (30) having a proximal end, a distal end and having at least one electrode mounted thereon. A lumen (32) extends through at least a portion of the elongate member (30) from an orifice (33) positioned at or relatively closer to the proximal end than the distal end. The lumen (32) is able to receive a stiffening element (34) through the orifice (33). The lumen (32) can be closed by a seal (35) that is pierceable by the stiffening element (34) but which at least substantially seals the lumen following removal of the stiffening element therefrom. The lumen (32) can also be closed by a plug (50) or a sealing member (72) mounted on the stiffening member. The lumen (32) can also be closed by a crimp (90) or clip (100) that is mountable around the elongate member (30) and which can compress the lumen (32).

### FIELD OF THE INVENTION

The present invention relates to an implantable device and, in particular, to an implantable cochlear electrode assembly.

### BACKGROUND OF THE INVENTION

Hearing loss, which may be due to many different causes, is generally of two types, conductive and sensorineural. Of these types, conductive hearing loss occurs where the normal mechanical pathways for sound to reach the hair cells in the cochlea are impeded, for example, by damage to the ossicles. Conductive hearing loss may often be helped by use of conventional hearing aid systems, which amplify sound so that acoustic information does reach the cochlea and the hair cells.

In many people who are profoundly deaf, however, the reason for deafness is sensorineural hearing loss. This type of hearing loss is due to the absence of, or destruction of, the hair cells in the cochlea which transduce acoustic signals into nerve impulses. These people are thus unable to derive suitable benefit from conventional hearing aid systems, because there is damage to or absence of the mechanism for nerve impulses to be generated from sound in the normal manner.

It is for this purpose that cochlear implant systems have been developed. Such systems bypass the hair cells in the cochlea and directly deliver electrical stimulation to the auditory nerve fibres, thereby allowing the brain to perceive a hearing sensation resembling the natural hearing sensation normally delivered to the auditory nerve.

Cochlear implant systems have typically consisted of two components, namely an external component commonly referred to as a processor unit, and an implanted internal component commonly referred to as a receiver/stimulator unit. Traditionally, both of these components have cooperated together to provide the sound sensation to an implantee.

The external component has traditionally consisted of a microphone for detecting sounds, such as speech and environmental sounds, a speech processor that converts the detected sounds and particularly speech into a coded signal, a power source such as a battery, and an external antenna transmitter coil.

The coded signal output by the speech processor is transmitted transcutaneously to the implanted receiver/stimulator unit situated within a recess of the temporal bone of the implantee. This transcutaneous transmission occurs through use of an inductive coupling provided between the external antenna transmitter coil which is positioned to communicate with an implanted antenna receiver coil provided with the receiver/stimulator unit. This communication serves two essential purposes, firstly to transcutaneously transmit the coded sound signal and secondly to provide power to the implanted receiver/stimulator unit. Conventionally, this link has been in the form of a radio frequency (RF) link, but other such links have been proposed and implemented with varying degrees of success.

The implanted receiver/stimulator unit typically includes the antenna receiver coil that receives the coded signal and power from the external processor component, and a stimulator that processes the coded signal and outputs a stimulation signal to an intracochlear electrode assembly which applies the electrical stimulation directly to the auditory nerve producing a hearing sensation corresponding to the original detected sound.

Several procedures have been adopted to provide an electrode assembly that can be inserted into the cochlea. In one case, a straight platinum wire stylet is positioned within a lumen extending along at least a portion of the length of the assembly. The stylet is relatively stiffer than the body of the assembly and serves to hold a pre-curved electrode array in a generally straight configuration up until insertion. Following insertion, the platinum stylet is withdrawn from the lumen allowing the array to return to its pre-curved configuration.

The presence of any lumen within the electrode assembly for the stylet may pose a potential pathway for pathogens including harmful bacteria, to migrate from a location external the cochlea into the cochlea if there is an opening from the lumen into the cochlea. While most implants are typically designed and constructed to ensure there is no potential pathway, other circumstances may dictate that such a lumen or an opening from such a lumen is desirable. The present invention provides a mechanism that could prevent any potential migration of pathogens through the assembly.

While the above description of the prior art is directed to cochlear implant electrode assemblies, similar issues of potential pathogen migration arise in other implantable devices using electrode assemblies, such as midbrain implants and muscle stimulation systems used in function electronic stimulation (FES) systems.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

### SUMMARY OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

According to a first aspect, the present invention is an implantable tissue-stimulating device comprising:

a resiliently flexible elongate member having a proximal end, a distal end, and having at least one electrode mounted thereon;

a lumen extending through at least a portion of the elongate member from an orifice positioned at or relatively closer to the proximal end than the distal end, the lumen being able to receive a stiffening element through the orifice; and

a seal that is pierceable by the stiffening element but which at least substantially seals the lumen following removal of the stiffening element therefrom.

According to a second aspect, the present invention is an implantable tissue-stimulating device comprising:

a resiliently flexible elongate member having a proximal end, a distal end, and having at least one electrode mounted thereon;

a lumen extending through at least a portion of the elongate member from an orifice positioned at or relatively closer to the proximal end than the distal end;

a stiffening element extending through at least a portion of the lumen and out through the orifice; and

a seal that at least substantially seals the lumen following removal of the stiffening element therefrom.

In one embodiment, the seal can be positioned in the lumen at or adjacent the orifice thereof. In one embodiment, the seal can extend over the orifice and be entirely external the lumen. In a still further embodiment, the seal can be positioned at the orifice. In this embodiment, the seal can extend partially outside the orifice and partially inside the orifice. In one embodiment, the seal can be essentially flush or flush with the orifice in the lumen.

In a still further embodiment, the seal can be formed from a resilient material. In this embodiment, the resilience of the seal can be sufficient such that on removal of the stiffening element therefrom, the seal closes across the passage formed in the seal by the placement of the stiffening element. In one embodiment, a slit can be formed in the seal that facilitates the passage of the stiffening element through the seal. Again, the resilience of the seal can be such that the slit is closed on removal of the stiffening element therefrom.

In yet a further embodiment, the seal can be formed of a silicone polymer. In one embodiment, a suitable portion of silicone polymer can be placed over or in the orifice of the lumen. In this embodiment, the drop cures and forms a meniscus-shaped layer over the lumen orifice. In another embodiment, a separately moulded seal can be formed and then adhered to the elongate member over the orifice. In this case, the seal can have a diameter greater than the orifice (eg. a diameter of about or greater than 0.18 mm) and a thickness of about 0.2 mm.

According to a third aspect, the present invention is a method of manufacturing an implantable tissue-stimulating device as defined herein with reference to the first or second aspects, the method comprising the steps of:

(i) sealing the lumen of the elongate member with a pierceable seal; and

(ii) piercing the seal with a tip of a stiffening element and sliding the stiffening element relatively into the lumen of the elongate member.

According to a fourth aspect, the present invention is a method of manufacturing an implantable tissue-stimulating device as defined herein with reference to the first or second aspects, the method comprising the steps of:

(i) positioning a stiffening element within a lumen of an elongate member, the stiffening element extending from within the lumen back out through an orifice of the lumen; and

(ii) sealing the orifice of the lumen of the elongate member with a seal.

According to a fifth aspect, the present invention is a method of placing an implantable tissue-stimulating device as defined herein with reference to the first and second aspects in the body of an implantee, the method comprising the steps of:

(i) inserting the elongate member into a desired location in the body of the implantee;

(ii) during and/or after insertion of the elongate member, at least partially relatively withdrawing the stiffening element from the lumen through the seal; and

(iii) allowing the seal to at least substantially seal the lumen of the elongate member.

In one embodiment of the fifth aspect, the stiffening element can be fully withdrawn from the lumen in step (ii). In another embodiment, the stiffening element can be only partially withdrawn from the lumen.

According to a sixth aspect, the present invention is an implantable tissue-stimulating device comprising:

a resiliently flexible elongate member having a proximal end, a distal end, and having at least one electrode mounted thereon;

a lumen extending through at least a portion of the elongate member from an orifice positioned at or relatively closer to the proximal end than the distal end, the lumen being able to receive a stiffening element through the orifice; and

a plug member that is positionable within and seals the orifice of the lumen following withdrawal of the stiffening element therefrom.

According to a seventh aspect, the present invention is a plug member that is positionable within and able to seal an orifice of a lumen of an elongate member of an implantable tissue-stimulating device.

In the seventh aspect, the elongate member can be resiliently flexible and have a proximal end and a distal end and at least one electrode mounted thereon. The lumen can extend through at least a portion of the elongate member from the orifice that is positioned at or relatively closer to the proximal end than the distal end. The plug can be positionable within the orifice following withdrawal of a stiffening element from the lumen. In one embodiment, the stiffening element is normally positioned within at least a portion of the lumen and extends out through the orifice.

According to an eighth aspect, the present invention is a method of placing an implantable tissue-stimulating device as defined herein with reference to the sixth aspect in the body of an implantee, the method comprising the steps of:

(i) inserting the elongate member into a desired location in the body of the implantee;

(ii) during and/or after insertion of the elongate member, relatively withdrawing the stiffening element from the lumen through an orifice in the member; and

(iii) inserting a plug into the orifice to at least substantially seal the lumen of the elongate member.

In one embodiment of the sixth, seventh and eighth aspects, the plug can be formed from a resiliently flexible material. In another embodiment, the plug can be formed from a relatively stiff material. For example, the plug can be formed from a biocompatible metallic material. In one embodiment, the plug can be formed from platinum or titanium.

The plug can be formed separately from the elongate member and positioned in the orifice to seal the lumen during or following placement of the elongate member in the implantee. The placement of the plug can be performed by the surgeon placing the elongate member in the implantee.

In one embodiment of the sixth, seventh and eighth aspects, the orifice of the lumen can be modified to suit and match with the construction of the plug. In another embodiment, the orifice of the lumen can be a standard tubular orifice with the plug formed to seal such an orifice on insertion therein.

In one embodiment of the sixth, seventh and eighth aspects, the plug can have a frusto-conical tapering portion. The frusto-conical portion can extend over a portion of the length of the plug or all of the length of the plug. In one embodiment, the plug can have a base member with an engaging portion extending outwardly therefrom. The engaging portion can have at least a portion having a diameter of between about 0.1 and 0.3 mm. Other dimensions to suit the dimensions of the orifice of the lumen can be envisaged. In a further embodiment, the engaging portion can have a length of between about 0.1 and 5 mm. Where present, the length of the base member can be less than that of the engaging portion. The base member can have a diameter greater than that of the engaging portion. Where the engaging portion has a diameter of about 0.18 mm, the base member can have a diameter of about 0.4 mm.

In a still further embodiment of the sixth, seventh and eighth aspects, the base member can include a grip member. The grip member can extend out of the base member in a direction opposite to that of the engaging portion. The grip member can be manipulable by a pair of forceps so allowing a surgeon to more readily place the plug in the orifice of the lumen of the elongate member. In one embodiment, the grip member can have a length of about 5mm.

According to a ninth aspect, the present invention is an implantable tissue-stimulating device comprising:

a resiliently flexible elongate member having a proximal end, a distal end, and having at least one electrode mounted thereon;

a lumen extending through at least a portion of the elongate member from an orifice positioned at or relatively closer to the proximal end than the distal end;

a stiffening element positioned at least partially within the lumen and extending out of the lumen through said orifice; and

a sealing member mountable to the stiffening element;

wherein the stiffening element is movable relative to the orifice of the lumen between a first position in which the sealing member mountable thereon does not seal the lumen and a second position in which the sealing member at least substantially seals the lumen.

According to a tenth aspect, the present invention is a stiffening element that is positionable through an orifice and into a lumen of an elongate member of a tissue-stimulating device, tie stiffening element comprising:

a stiffening member; and

a sealing member mountable to the stiffening member;

wherein the stiffening element is movable relative to the orifice of the lumen between a first position in which the sealing member mountable thereon does not seal the lumen and a second position in which the sealing member at least substantially seals the lumen.

In an embodiment of the ninth and tenth aspects, the sealing member can comprise a sealing portion of a resiliently flexible material mounted to the stiffening member of the stiffening element. The material can be movable relative to the stiffening member. In one embodiment, the material can comprise a biocompatible silicone. In this and other embodiments, the sealing portion can be cylindrical in form.

During manufacture and/or placement of the stiffening member in the lumen of the elongate member, the portion of resiliently flexible material can be initially mounted adjacent a distal end of the stiffening member and within the lumen of the elongate member, i.e. a first position. On relative removal of the stiffening member from the lumen, the sealing portion can remain mounted to the stiffening member and is drawn through the lumen towards the orifice of the lumen. In a further embodiment, the orifice of the lumen is smaller in diameter than at least the majority of the lumen. As the stiffening member relatively withdraws, the sealing portion eventually abuts the inner wall of the lumen where it narrows to form the orifice. In one embodiment, this abutment of the sealing portion can act to at least substantially seal the orifice. In another embodiment, the sealing portion can be at least partially drawn into the narrowing of the lumen and so form a seal therewith. In these embodiments, with the sealing member now in the second position, further withdrawing force on the stiffening member can serve to disengage the stiffening member from the sealing portion so allowing the stiffening member to be fully withdrawn through the orifice.

In another embodiment, the sealing portion can be non-removably mounted at or relatively near the distal end of the stiffening member. In this case, on relative withdrawal of the stiffening member from the lumen, the sealing portion is again carried into sealing abutment or engagement with the narrowing of the lumen. Once in this position, the stiffening member can be severed at or adjacent the orifice so leaving the sealing portion with a relatively short portion of the stiffening member embedded therein in the orifice of the lumen.

In a still further embodiment, the sealing member can be an integral part of the stiffening member.

In yet another embodiment, the sealing member can have a shape that matches the shape of the narrowing of the lumen at or adjacent the orifice thereof. For example, the narrowing of the lumen can comprise a frusto-conical region with the lumen narrowing towards the orifice. In this embodiment, the sealing member can also be at least partially frusto-conical in form and shaped to match the taper of the narrowing portion of the lumen.

In the above embodiments, the sealing member can be formed of a material different to that making up the elongate member. Where the elongate member is made of a silicone, the sealing member can be formed of a material having a relatively lower coefficient of friction. For example, the material can be formed of an epoxy material, platinum, iridium and/or can have a parylene coating. The sealing member can also have a lubricious coating.

In a still further embodiment, the sealing member can comprise a substantially spherical or spherical member mounted at or relatively near the distal end of the stiffening member. In another embodiment, the member can have another shape. Where the stiffening member is a metal stylet, as is defined in more detail below, the sealing member can be a platinum sphere (or a plastic sphere) mounted to the distal end of the stylet. The sphere can be integrally or non-integrally mounted to the distal end. In this embodiment, the diameter of the stylet can be about 0.125 mm, while the sphere can have a diameter larger than the stylet, for example about 0.15 mm.

In this embodiment, the narrowing of the lumen adjacent the orifice can be shaped to receive the sphere at the end of the stylet. In this regard, the region can comprise a spherical region shaped to receive the sphere. In one embodiment, the spherical region can have a diameter less than that of the sphere of the stylet. For example, where the sphere has a diameter of about 0.15 mm or more, the spherical region can have a diameter of about 0.12 mm or less. The spherical region can be located a relatively short distance from the orifice of the lumen. In one embodiment, the spherical region can be spaced a distance of about 0.3 mm from the orifice. In this region, the lumen can comprise a cylindrical region having a diameter less than that of the spherical region, for example, 0.1 mm or less. A further cylindrical region of the lumen can also extend from the spherical region into the elongate member before the lumen expands to a larger diameter. The further cylindrical region can have a diameter of about 0.1 mm and a length of about 0.2 mm or more. The remainder of the lumen can have a diameter of about 0.18 mm for at least a majority of its length.

In this embodiment, it will be appreciated that as the surgeon relatively withdraws the stylet from the lumen, the surgeon will note the increase in friction as the sphere enters the smaller diameter cylindrical region on the distal side of the spherical region. On noticing this increase, the surgeon would be aware to slow the rate of relative withdrawal until the surgeon notes that the sphere has entered the spherical region. At this time, the surgeon would stop withdrawal and trim the stylet at or adjacent the orifice. The presence of the sphere positioned in the spherical region serves to at least substantially seal the orifice of the lumen.

According to an eleventh aspect, the present invention is a method of placing an implantable tissue-stimulating device as defined herein with reference to the ninth aspect in the body of an implantee, the method comprising the steps of:

(i) inserting the elongate member into a desired location in the body of the implantee;

(ii) during and/or after insertion, at least partially relatively withdrawing the stiffening element from the lumen through the orifice; and

(iii) bringing the sealing member that is mountable to the stiffening element into a position in which the sealing member at least substantially seals the lumen.

According to a twelfth aspect, the present invention is an implantable tissue-stimulating device comprising:

a resiliently flexible elongate member having a proximal end, a distal end, and having at least one electrode mounted thereon;

a lumen extending through at least a portion of the elongate member from an orifice positioned at or relatively closer to the proximal end than the distal end; and

a compression member mountable around at least a portion of the elongate member;

wherein the compression member is adjustable between a first configuration in which the compression member does not compress a portion of the lumen and a second configuration in which the compression member does compress at least a portion of the lumen.

In one embodiment of the twelfth aspect, the lumen is able to receive a stiffening element through the orifice thereof. The lumen of the elongate member can be the same diameter along at least a majority or all of its length. In another embodiment, the lumen can have a region of lesser diameter at or adjacent the orifice of the lumen. In one embodiment, the compression member can be positioned around the elongate member at the region of lesser diameter. In one embodiment, the position of the compression member around the elongate member can be adjustable. An indicator can be provided to allow external identification of the location of the region of lesser diameter. Such an indicator may include a visual indicator.

In a further embodiment, the compression member can be only adjustable once from the first configuration to the second configuration. As such, once the compression member has been adjusted to adopt a second configuration, it remains in that configuration. Adjustment of the configuration can be performed by the surgeon during the implantation procedure for the elongate member. Where a stiffening element is positioned in the lumen, the stiffening element can be withdrawn during the implantation procedure. Following withdrawal of the stiffening element, the surgeon or another assisting in the surgical procedure can adjust the crimp and so compress at least a portion of the lumen.

The compression member can be formed from a biocompatible material. In one embodiment, the compression member can comprise a ring of material. The ring can be formed of metallic material, such as stainless steel, titanium, or a suitable alloy. In another embodiment, the compression member can be formed from a biocompatible plastics material, such as ABS or polypropylene. In these embodiments, the compression member can be in the form of a crimp. The crimp can be compressed flat or in another configuration, such as a zig-zag pattern. A compressing tool shaped to suit the dimensions of the crimp could be utilised in this regard.

In another embodiment, the compression member can comprise a clip which is mountable around the elongate member and which can then be closed and latched. The dimensions of the clip relative to the elongate member can be such that on closing and latching of the clip, at least a portion of the lumen is compressed sufficiently to at least substantially seal the lumen. The clip can be capable of manipulation by the hands of the surgeon. A suitable tool for performing this function can, however, also is envisaged. Such a clip can be formed from a biocompatible plastics material, such as polypropylene.

According to a thirteenth aspect, the present invention is an implantable tissue-stimulating device comprising:

a resiliently flexible elongate member having a proximal end, a distal end, and having at least one electrode mounted thereon; and

a lumen extending through at least a portion of the elongate member from an orifice positioned at or relatively closer to the proximal end than the distal end, said lumen being able to receive a stiffening element through the orifice;

wherein the lumen has at least one first portion of a first diameter and at least one second portion having a diameter less than that of said at least one first portion;

wherein said second portion is relatively closer to the orifice of the lumen than at least one of said first lumen portions.

The second portion of the lumen can have a diameter that is about the same or less than the diameter of the stiffening element passing therethrough. In one embodiment, the diameter of the second portion can be about 0.1 mm. The diameter of the first portion can be about 0.18 mm. In yet another embodiment, the second portion can have a length of about 5 mm.

In one embodiment, the second portion can be spaced from the orifice of the lumen by at least one first portion.

In a further embodiment, the second portion can be compressed by a compression member as defined herein according to the twelfth aspect of the invention. In another embodiment, the orifice of the lumen can have a quantity of adhesive inserted therein that subsequently cures and seals the lumen. The adhesive can be a cyanoacrylate.

In the thirteenth aspect, at least the second portion of the lumen can be coated with a material that swells on contact with at least certain fluids. For example, the second portion of the lumen can have a coating of a hydrogel material that swells following contact with body fluids. The hydrogel can have a coating to allow control of the commencement time or rate of swelling of the hydrogel. The swelling of the hydrogel can be such that the second portion is sealed following withdrawal of the stiffening element.

According to a fourteenth aspect, the present invention is an implantable tissue-stimulating device comprising:

a resiliently flexible elongate member having a proximal end, a distal end, and having at least one electrode mounted thereon; and

a lumen extending through at least a portion of the elongate member from an orifice positioned at or relatively closer to the proximal end than the distal end, said lumen being able to receive a stiffening element through the orifice;

wherein at least a portion of the lumen is coated with a layer of material that swells following exposure to bodily fluids.

According to a fifteenth aspect, the present invention is an implantable tissue-stimulating device comprising:

a resiliently flexible elongate member having a proximal end, a distal end, and having at least one electrode mounted thereon; and

a lumen extending through at least a portion of the elongate member from an orifice positioned at or relatively closer to the proximal end than the distal end, said lumen being able to receive a stiffening element through the orifice;

wherein the lumen in the region adjacent the orifice decreases in diameter away from the orifice into the elongate member for a length.

In the fifteenth aspect, said region of the lumen can be frusto-conical in form. The shape and dimensions of said region can be such that at least part of the region can be packed with fibrous tissue following withdrawal of the stiffening element.

In a further embodiment of all of the aspects, the device is a cochlear implant electrode assembly. In another embodiment, the device can deliver stimulation to the brain, such as the midbrain. Still further, the device can deliver functional electrical stimulation to one or more muscle groups in the body of an implantee.

In a further embodiment of all of the aspects, the distal end of the elongate member can be insertable firstly into the implantee.

The lumen can be circular in cross-section or have any other suitable cross-sectional shape. In one embodiment, the lumen extends through the elongate member for a substantial portion of its length. In a further embodiment, the lumen extends from an opening at the proximal end of the elongate member to a position that is adjacent the distal end thereof. In one embodiment, the shape and/or diameter of the lumen at or adjacent the orifice can be different to that of the remainder of the lumen. For example, the diameter of the lumen can gradually decrease in diameter from the orifice inwardly for a portion of the length of the lumen.

In a further embodiment, the elongate member can have a plurality of electrodes mounted thereon. In one embodiment, the electrodes can be formed of a biocompatible metallic material, such as platinum.

In a further embodiment, the elongate member can have a first configuration selected to allow said member to be more readily inserted into an implantee's body, such as the cochlea, and a second configuration wherein said elongate member is more readily able to apply a preselected tissue stimulation with the electrodes. In a further embodiment, the elongate member can have at least one intermediate configuration between said first and second configurations.

In a still further embodiment, at least a portion of the outer surface of the elongate member can have a coating of lubricious material. In a further embodiment, a substantial portion of the outer surface can have a coating of the lubricious material. In a still further embodiment, the entire outer surface of the elongate member can have a coating of the lubricious material.

The lubricious material becomes lubricious on being brought into contact with a fluid, such as a saline solution. Still further, the coating becomes lubricious on being brought into contact with a body fluid, such as cochlear fluid.

In one embodiment, the lubricious material is selected from the group comprising polyacrylic acid (PAA), polyvinyl alcohol (PVA), polylactic acid (PLA) and polyglycolic acid (PGA). It is envisaged that other similar materials could also be used.

As already defined with reference to some aspects, in yet another embodiment, the device can include a stiffening element made of a second material relatively stiffer than the resiliently flexible material of the elongate member. The stiffening element can bias the elongate member into the first configuration.

In one embodiment, the second configuration of the elongate member is curved. Still further, the elongate member can adopt a spiral configuration when in the second configuration.

The elongate member can be preformed from a plastics material with memory and be preformed to the second configuration.

In one embodiment, the first configuration is substantially straight. Still further, the first configuration can be straight.

In yet another embodiment, the elongate member is formed from a suitable biocompatible material. In one embodiment, the material can be a silicone, such as Silastic MDX 4-4210. In another embodiment, the elongate member can be formed from a polyurethane or similar material.

In still another embodiment, the stiffening element can be formed from a non-bioresorbable material. In this embodiment, the stiffening element can comprise a metallic stylet, or a stylet-like element formed from any other suitable stiffening material, extending through a lumen in the elongate member. In one embodiment, the stylet can be formed from a biocompatible metal, a biocompatible metallic alloy or a biocompatible relatively stiff plastic. In one embodiment, a metal stylet can be formed from platinum.

In the case of a metal stylet, the stylet can extend out of the orifice through the seal allowing the stylet to be manipulated and removed from the lumen during or following insertion of the device.

Once implanted, the electrodes can receive stimulation signals from a stimulator device. The stimulator device can be electrically connected to the elongate member by way of an electrical lead. The lead can include the one or more wires extending from each electrode of the array mounted on the elongate member.

In one embodiment, the lead can extend from the elongate member to a stimulator device or at least the housing thereof. In one embodiment, the lead is continuous with no electrical connectors, at least external the housing of the stimulator device, required to connect the wires extending from the electrodes to the stimulator device. One advantage of this arrangement is that there is no requirement for the surgeon implanting the device to make the necessary electrical connection between the wires extending from the electrodes and the stimulator device.

The stimulator device can be positioned within a housing that is implantable within the implantee. The housing for the stimulator device can be implantable within the bony well in the bone behind the ear posterior to the mastoid.

When implantable, the housing can contain, in addition to the stimulator device, a receiver device. The receiver device can receive signals from a controller. The controller is, in use, mountable external to the body of the implantee such that the signals are transmitted transcutaneously through the implantee.

Signals can travel from the controller to the receiver device and vice versa. The receiver device can include a receiver coil that receives radio frequency (RF) signals from a corresponding transmitter coil worn externally of the body. The radio frequency signals can comprise frequency modulated (FM) signals. While described as a receiver coil, the receiver coil can also transmit signals to the transmitter coil which receives the signals.

The transmitter coil can be held in position adjacent the implanted location of the receiver coil by way of respective attractive magnets mounted centrally in, or at some other position relative to, the coils.

The external controller can comprise a speech processor able to receive signals output by a microphone. During use, the microphone can be worn on the pinna of the implantee, however, other suitable locations can be envisaged, such as a lapel of the implantee's clothing. The speech processor encodes the sound detected by the microphone into a sequence of electrical stimuli following given algorithms, such as algorithms already developed for cochlear implant systems. The encoded sequence is transferred to the implanted receiver/stimulator device using the transmitter and receiver coils. The implanted receiver/stimulator device demodulates the FM signals and allocates the electrical pulses to the appropriate attached electrode by an algorithm which is consistent with the chosen speech coding strategy.

The external controller further comprises a power supply. The power supply can comprise one or more rechargeable batteries. The transmitter and receiver coils are used to provide power via transcutaneous induction to the implanted receiver/stimulator device and the electrode array.

While the implant system can rely on external componentry, in another embodiment, the controller, including the microphone, speech processor and power supply can also be implantable. In this embodiment, the controller can be contained within a hermetically sealed housing or the housing used for the stimulator device.

### BRIEF DESCRIPTION OF THE DRAWINGS

By way of example only, embodiments of the invention are now described with reference to the accompanying drawings, in which:

FIG. 1 is a pictorial representation of a prior art cochlear implant system;

FIG. 2a is a fragmentary view of a portion of an elongate member having one embodiment of a seal according to the present invention;

FIG. 2b is a fragmentary view of a portion of an elongate member having another embodiment of a seal according to the present invention;

FIG. 3 is a fragmentary view of the elongate member of FIG. 2a with a stylet in position in the elongate member;

FIG. 4a is a perspective view of one embodiment of a seal for use in the invention;

FIG. 4b is a perspective view of an elongate member depicting the seal of FIG. 4a in position in the orifice of the lumen of the elongate member;

FIG. 5 is a fragmentary view of a portion of an elongate member having one embodiment of a lumen according to the present invention;

FIGS. 6a to 6d are side views of plugs for placement in the orifice of the lumen of one embodiment of a elongate member according to the present invention;

FIG. 7 is a fragmentary view of a portion of a stylet having a sealing member movably mounted thereto;

FIG. 8 is a fragmentary view of an elongate member having a sealing member positioned within the lumen following withdrawal of the stylet of FIG. 7;

FIG. 9 is a fragmentary view of an elongate member having a sealing member mounted to a severed stylet and positioned at least adjacent the orifice of the lumen;

FIG. 10 is a fragmentary view of a further embodiment of the present invention;

FIG. 11a is a view of a stylet having a sphere at a distal end thereof;

FIG. 11b is a fragmentary view of an elongate member adapted for use with the stylet of FIG. 11a;

FIG. 11c depicts the stylet of FIG. 11a sealably positioned within the elongate member of FIG. 11b;

FIGS. 12 and 13 are diagrammatic views of a portion of an elongate member having one embodiment of a compression crimp according to the present invention mounted thereto;

FIGS. 14 and 15 depict the crimp of FIGS. 12 and 13 following different types of crimping;

FIGS. 16a and 16b depict one embodiment of a compression clip according to the present invention mounted thereto in an open and closed position, respectively; and

FIG. 17 is a fragmentary view of an elongate member having a lumen having a narrow region.

### PREFERRED MODE OF CARRYING OUT THE INVENTION

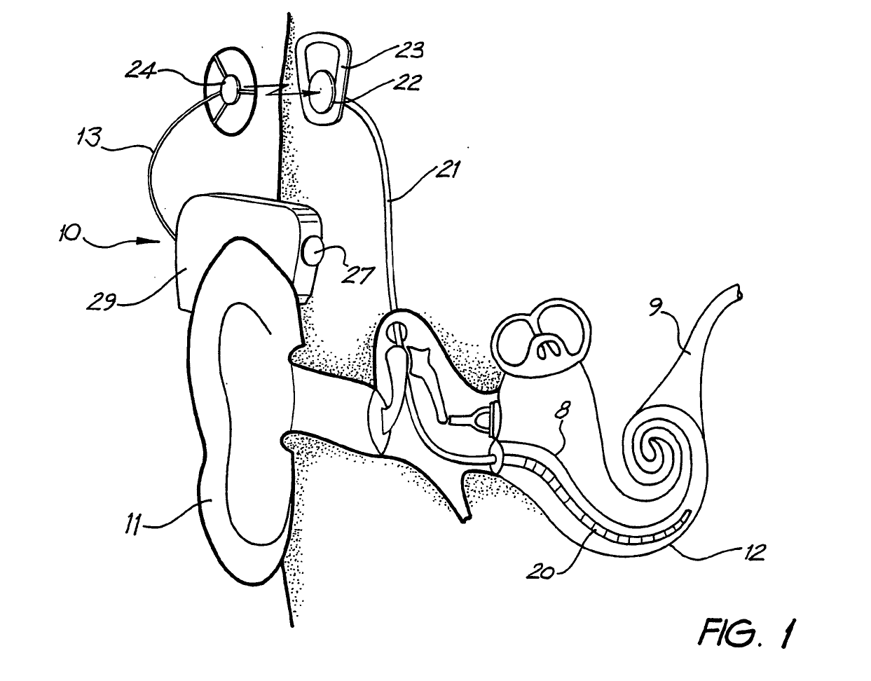

Before describing the features of the present invention, it is appropriate to briefly describe the construction of a typical cochlear implant system with reference to FIG. 1.

Cochlear implants typically consist of two main components, an external component including a speech processor 29, and an internal component including an implanted receiver and stimulator unit 22. The external component includes a microphone 27. The speech processor 29 is, in this illustration, constructed and arranged so that it can fit behind the outer ear 11. Alternative versions may be worn on the body. Attached to the speech processor 29 is a transmitter antenna coil 24 which transmits electrical signals to the implanted unit 22 via a radio frequency (RF) link.

The internal component includes a receiver antenna coil 23 for receiving power and data from the transmitter coil 24. A cable 21 extends from the implanted receiver and stimulator unit 22 to the cochlea 12 and terminates in an electrode array 20. The signals thus received are applied by the array 20 to the basilar membrane 8 and the nerve cells within the cochlea 12 thereby stimulating the auditory nerve 9. The operation of such a device is described, for example, in U.S. Pat. No. 4,532,930, the contents of which are incorporated herein by reference.

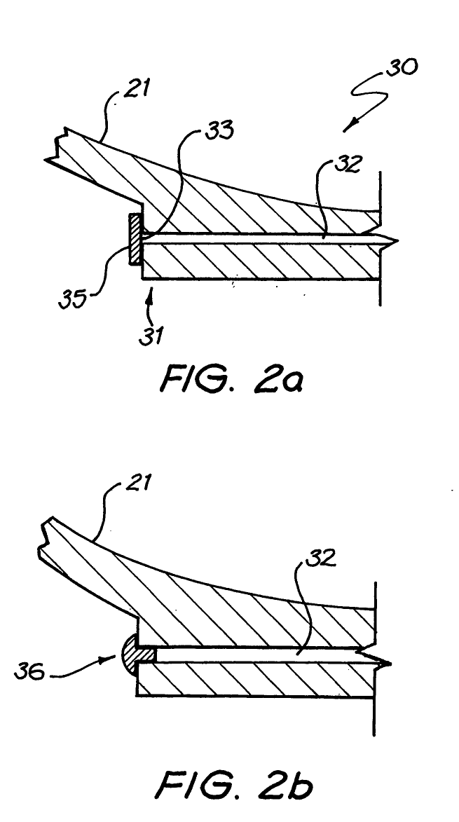

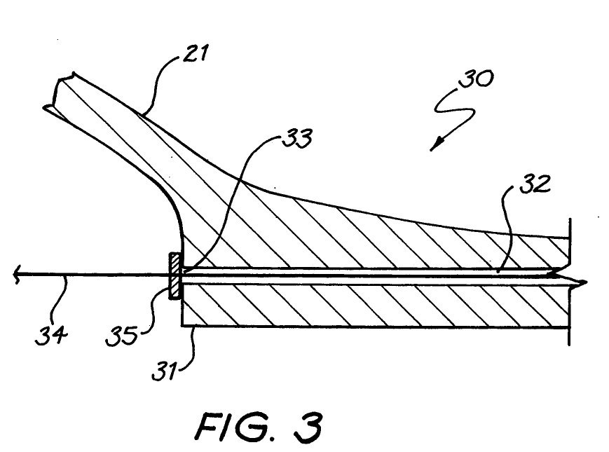

One embodiment of a cochlear implant electrode assembly according to the present invention is depicted generally as 30 in FIGS. 2a and 3. While the drawings are directed to cochlear implants, it will be appreciated that the present invention could be used in conjunction with other implantable tissue-stimulating devices such as devices for delivering stimulation to the brain, such as the midbrain, and devices that can deliver functional electrical stimulation to one or more muscle groups in the body of an implantee.

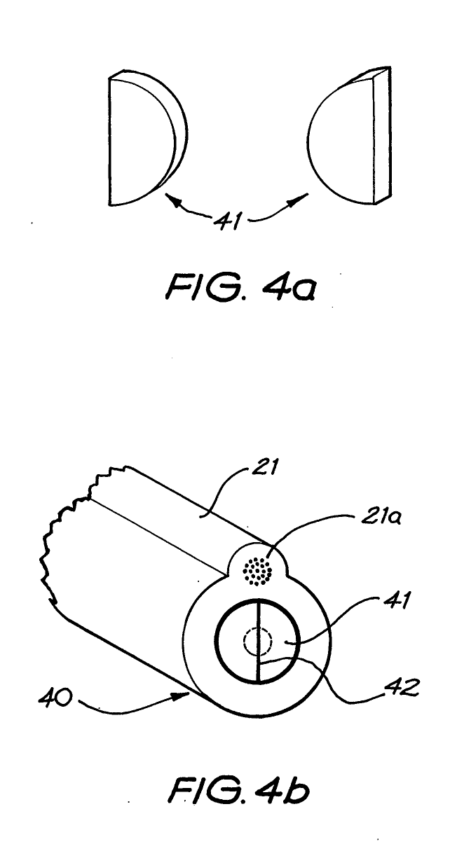

The elongate member 30 has a proximal end 31 and can carry a plurality of electrodes, which are not depicted in these drawings for reasons of clarity. A lead or cable 21 extends into the elongate member 30. One or more electrically conducting wires 21a (depicted in FIG. 4b) extend through the lead 21 from a stimulator unit to the respective platinum electrodes of the member 30.

The member 30 has a lumen 32 extending into and along at least a portion of the elongate member from an orifice 33. In the depicted embodiment, the lumen 32 is to be understood as extending to a location near the distal end (that is not visible) of the member 30. The distal end is normally the end of the member 30 that is firstly implanted into the implantee. The depicted lumen is circular in cross-section but it will be appreciated that other suitable cross-sectional shapes could be utilised. In a further embodiment, the lumen extends from an opening at the proximal end of the elongate member to a position that is adjacent the distal end thereof.

The lumen 32 is able to receive a stiffening element, such as a platinum stylet 34 which is depicted in FIG. 3.

The orifice 33 of the lumen 32 is depicted as being closed by a seal 35 in FIGS. 2a and 3. In these Figures, the seal 35 is external to and extends over the orifice 33. In FIG. 2b, an alternative seal structure is depicted as seal 36 in which the seal 36 extends from a position external the orifice 33 into the lumen 32. It will be appreciated that the seal could also be positioned entirely within the lumen 32 of the member. In one embodiment, the seal can be essentially flush or flush with the orifice 33 of the lumen 32.

In these embodiments, the seal is positioned at or adjacent the orifice and is then pierced by the stylet 34 so allowing the stylet 34 to remain in the lumen 32 as long as is required (see FIG. 3). It will be appreciated that the stylet 34 could be positioned in the lumen 32 during the manufacturing process for the member 30. In another embodiment, the stylet may not be inserted into the lumen and so used to straighten the elongate member until a time relatively close to the implantation of the member 30 into the cochlea of an implantee.

In a further embodiment, the stylet 34 can be positioned in the lumen 32 prior to the seal 35 being put in place to close the orifice 33. The result is the same as the alternative method of manufacture with the stylet 34 in the lumen 32 and the orifice sealed by seal 35 as depicted in FIG. 3.

In each depicted embodiment, the seal 35 is formed from a resilient silicone material. In one embodiment, a suitable drop or portion of silicone polymer can be placed over or in the orifice 33 of the lumen 32. In this embodiment, the drop cures and forms a meniscus-shaped layer over the lumen orifice 33. The resilience of the seal 35 is sufficient that on removal of the stylet 34 from the lumen 32, the seal 35 closes across the passage formed in the seal 35 by the placement of the stylet therethrough.

An alternative embodiment of a seal is depicted generally as 40 in FIG. 4b. In this embodiment, the seal is formed from two separately moulded D-shaped portions 41 that can be positioned side-by-side or overlapping in the orifice 33 of the lumen 32 of the elongate member. The positioning of the portions 41 in the orifice 33 results in a slit 42 being formed in the seal that facilitates the passage of the stylet through the seal. Again, the resilience of the seal is such that the slit 42 is closed on removal of the stylet therefrom.

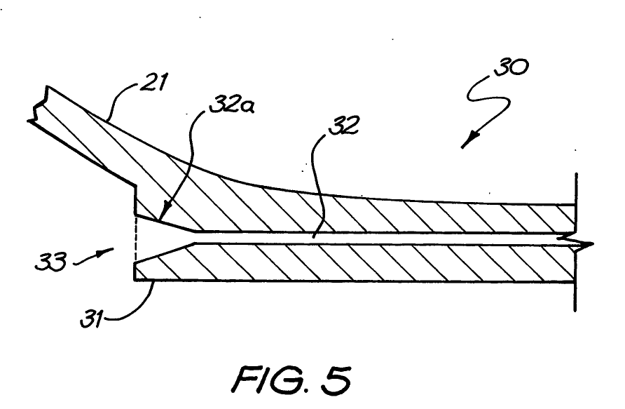

An alternative embodiment of an elongate member 30 having a lumen 32 extending into and along at least a portion of the elongate member from an orifice 33 is depicted in FIG. 5. In this embodiment, the lumen 32 is again cylindrical for a majority of its length, however, a portion 32a of the lumen adjacent the orifice 33 is frusto-conical in form with the diameter of the portion 32a decreasing away from the orifice 33 over its length.

The shape and dimensions of the region 32a are such that at least part of the region can be packed with fibrous tissue, if desired, following withdrawal of the stylet.

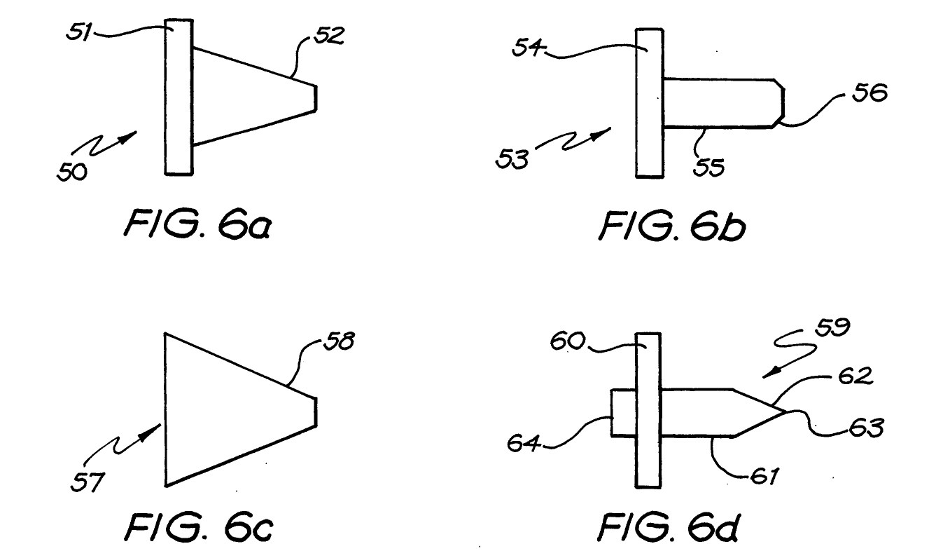

In addition or instead of this step and on implantation of the member 30 of FIG. 5 and withdrawal of the stylet, the orifice 33 of the lumen 32 can be sealed by a plug. Various forms of suitable plugs are depicted in FIGS. 6a to 6d.

In one embodiment, the plug used to seal the orifice 33 can be formed from a resiliently flexible material. In another embodiment, the plug can be formed from a relatively stiff material. For example, the plug can be formed from a biocompatible metallic material, such as platinum or titanium.

In each of the embodiments depicted in FIGS. 6a to 6d, the plug has a frusto-conical tapering portion.

As depicted in FIG. 6a, the plug 50 has a base member 51 with an engaging portion 52 extending outwardly therefrom. In this embodiment, the engaging portion 52 has a diameter of about 0.18 mm about midway along its length. Other dimensions to suit the dimensions of the orifice of the lumen can be envisaged. The depicted plug is of a diameter that is sufficient to cause at least a slight expansion of the orifice 33 so as to ensure that the plug is held tight within the orifice.

In FIG. 6b, the plug 53 again has a base member 54 and an engaging portion 55. In this embodiment, the engaging portion is cylindrical for a majority of its length but does have a tapering distal end 56. In this embodiment, the cylindrical portion of the engaging portion 55 has a diameter of about 0.18 mm, while the diameter of the base member is greater than the portion 55 and in the depicted embodiment is about 0.4 mm.

In FIG. 6c, the plug 57 simply comprises a frusto-conical engaging portion 58 that extends the length of the portion 58. In this embodiment, the engaging portion 58 has a diameter of about 0.18 mm about midway along its length.

In FIG. 6d, the plug 59 again has a base member 60 and an engaging portion 61. The engaging portion 61 has a tapering end 62 that tapers to a point 63. In this embodiment, the plug 59 has a further gripping portion 64 extending out of the base member in a direction opposite to that of the engaging portion 61. The gripping portion is designed to allow the plug to be readily graspable by a pair of forceps or the like and so facilitate placement of the plug in the orifice 33 of the lumen of the elongate member. In another embodiment, the engaging portion can have a waist separated from the proximal end to act as an impediment to the plug falling out of the orifice 33. In this regard, the engaging portion can have a smaller diameter section or tapered conical section on the plug immediately adjacent the proximal end.

In each embodiment, the engaging portion can have a length of between about 0.1 and 5 mm. Where present, the length of the base member is typically less than that of the engaging portion. The base member also has a diameter greater than that of the engaging portion.

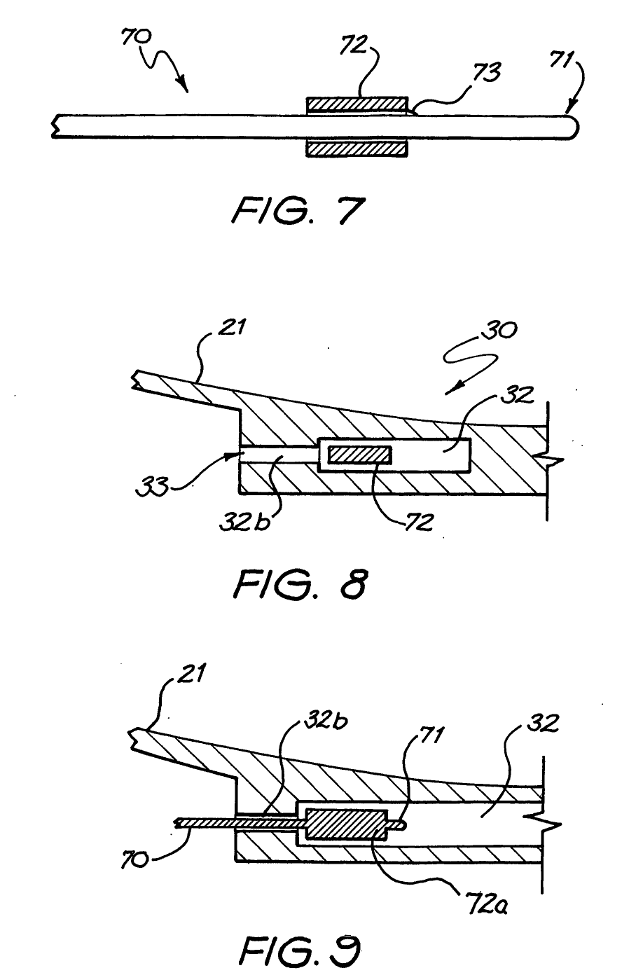

One embodiment of an alternative stylet according to the present invention is depicted generally as 70 in FIG. 7. The depicted stylet 70 is formed of platinum but it will be appreciated that other biocompatible metals and plastics materials or combinations thereof could be employed as a stylet in the present invention. The stylet 70 is relatively stiffer than the silicone elongate member 30 in FIG. 8 and is adapted to be positioned within a lumen 32 thereof and so straighten the elongate member 30 from its preferentially curved configuration to one that is more readily implantable in the cochlea of an implantee.

The stylet 70 has a distal end 71 and has, prior to its relative withdrawal from the lumen 32, a cylindrical resiliently flexible sealing portion 72 that is tethered thereto through tether 73. During manufacture and/or placement of the stylet 70 in the lumen 32 of the elongate member 30, the sealing portion 72 is initially mounted adjacent the distal end 71 and within the lumen 32 of the elongate member, i.e. a first position. On relative removal of the stylet 70 from the lumen 32, the sealing portion 72 remains tethered to the stylet and is drawn through the lumen 32 towards the orifice 33 thereof. As depicted, the lumen 32 has a relatively narrow portion 32b adjacent the orifice 33. As the stylet 70 relatively withdraws, the sealing portion 72 eventually abuts the inner wall of the lumen where it narrows adjacent the orifice, i.e. narrow region 32b. This abutment of the sealing portion 72 acts to at least substantially seal the orifice 33.

In another embodiment, the sealing portion 72 can be at least partially drawn into the narrow region 32b of the lumen and so form a seal therewith. In these embodiments, with the sealing portion 72 now in the second position, further withdrawing force on the stylet 70 serves to disengage the stylet 70 from the sealing portion 72 so allowing the stylet 70 to be fully withdrawn through the orifice 33.

In another embodiment depicted in FIG. 9, the sealing portion (here depicted as 72a) can be non-removably mounted at or relatively near the distal end 71 of the stylet 70. In this case, on relative withdrawal of the stylet 70 from the lumen 32, the sealing portion 72a is again carried into sealing abutment or engagement with the narrow region 32b of the lumen. Once in this position, the stylet 70 can be severed at or adjacent the orifice 33 so leaving the sealing portion 72a with a relatively short portion of the stylet 70 embedded therein in the narrow region 32b of the lumen.

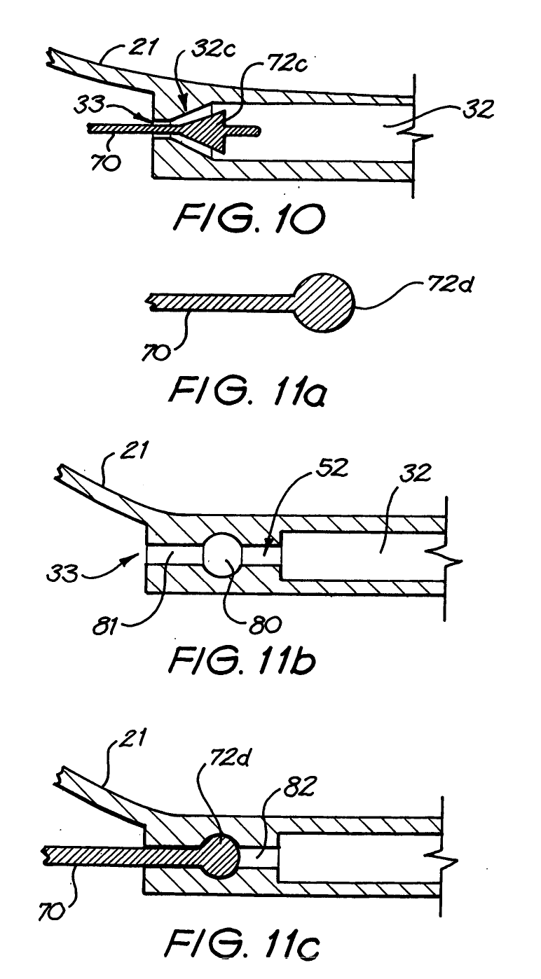

In a still further embodiment depicted in FIGS. 10 to 11c, the sealing member can be an integral part of the stylet.

In FIG. 10, the sealing member 72c has a shape that matches the shape of the narrow region 32c of the lumen at or adjacent the orifice 33 thereof. As depicted, the narrow region 32c comprises a frusto-conical region with the lumen narrowing towards the orifice 33. In this embodiment, the sealing member 72c is also at least partially frusto-conical in form and shaped to match the taper of the narrow region 32c.

In these embodiments, the sealing members can be formed of a material different to that making up the elongate member. Where the elongate member is made of a silicone, the sealing member is formed of a material having a relatively lower coefficient of friction. For example, the material can be an epoxy material, platinum, iridium and/or can have a parylene or lubricious coating.

As depicted in FIGS. 11a to 11c, the sealing member can comprise a spherical member 72d mounted at the distal end of a stylet 70. Where the stylet 70 is a metal stylet, the sealing member can be a platinum sphere mounted to and/or integral with the distal end of the stylet. The depicted sphere 72d is integrally mounted to the distal end of the stylet 70. In this embodiment, the diameter of the stylet can be about 0.125 mm, while the sphere can have a diameter of about 0.15 mm.

In this embodiment, the narrowing of the lumen 32 adjacent the orifice is shaped to receive the sphere 72d at the end of the stylet 70. In this regard, the region comprises a spherical region 80 shaped to receive the sphere 72d. In this embodiment, the spherical region 80 has a diameter less than that of the sphere 72d. For example, where the sphere 72d has a diameter of about 0.15 mm, the spherical region 80 can have a diameter of about 0.12 mm. The spherical region 80 is located a relatively short distance from the orifice 33. In the depicted embodiment, the spherical region 80 is spaced a distance of about 0.3 mm from the orifice 33. In this region, the lumen can comprise a cylindrical region 81 having a diameter less than that of the spherical region 80, for example, 0.1 mm. A further cylindrical region 82 of the lumen also extends from the spherical region 80 into the elongate member before the lumen expands to a larger diameter. The further cylindrical region 82 can have a diameter of about 0.1 mm and a length of about 0.2 mm. The remainder of the lumen has a diameter of about 0.18 mm for at least a majority of its length.

In this embodiment, it will be appreciated that as the surgeon relatively withdraws the stylet 70 from the lumen, the surgeon will note the increase in friction as the sphere enters the smaller diameter cylindrical region 82 on the distal side of the spherical region 80. On noticing this increase, the surgeon would be aware to slow the rate of relative withdrawal until the surgeon feels that the sphere 72d has entered the spherical region 80. One or more indicators could also be provided on the stylet to indicate to the surgeon to slow the withdrawal when the indicators come out of the proximal end of the lumen. At this time, the surgeon would stop withdrawal and trim the stylet 70 at or adjacent the orifice. The presence of the sphere 72d positioned in the spherical region 80, as depicted in FIG. 11c, serves to at least substantially seal the lumen.

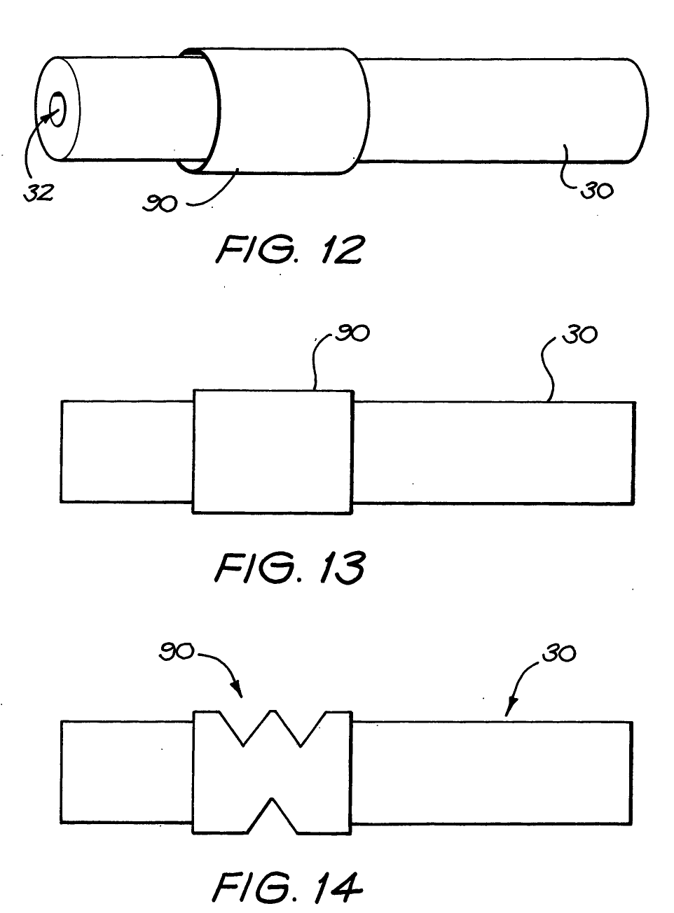

One embodiment of a compression member for use in sealing the lumen 32 of an elongate member 30 is depicted generally as 90 in FIGS. 12 and 13. It will be understood that the elongate member 30 can have a plurality of electrodes mounted thereon.

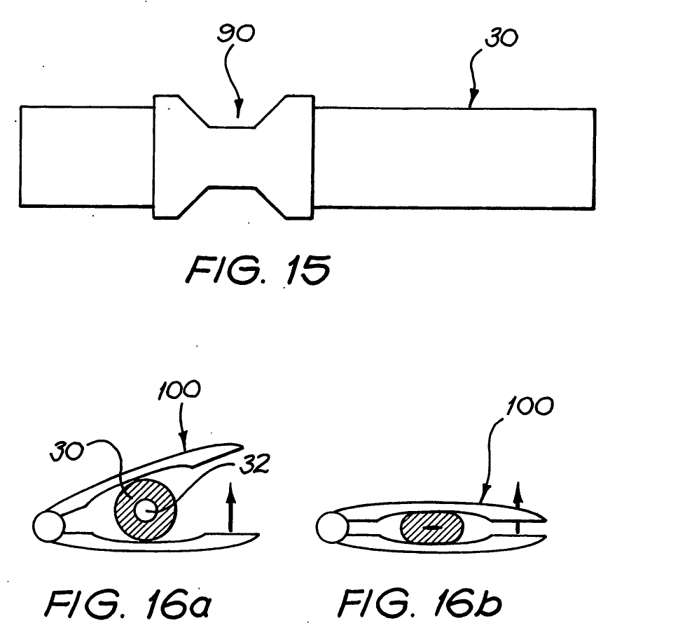

The compression member 90 comprises a ring that is mountable around at least a portion of the elongate member 30. The member 90 is adjustable between a first configuration (as depicted in FIG. 13) in which the compression member does not compress a portion of the lumen 32 and a second configuration (as depicted, for example, in FIGS. 14 and 15) in which the compression member does compress at least a portion of the lumen.

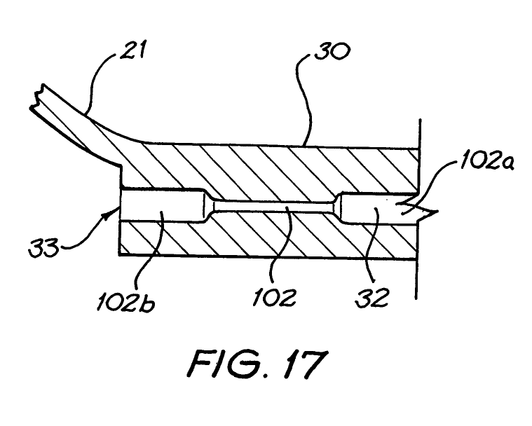

The compression member 90 can be provided on the elongate member at a location where the lumen 32 of the elongate member has a region 102 of lesser diameter as depicted in FIG. 17. An indicator can be provided to allow external identification of the location of the region 102 of lesser diameter. Such an indicator may include a visual indicator.

In the depicted embodiment, the compression member 90 is only adjustable once from the first configuration to the second configuration. As such, once the compression member has been adjusted to adopt a second configuration (as depicted in FIGS. 14 and 15), it remains in that configuration. Adjustment of the configuration is performed by the surgeon during the implantation procedure for the elongate member. Where a stylet is positioned in the lumen, the stylet is withdrawn during the implantation procedure. Following withdrawal of the stylet, the surgeon or another assisting in the surgical procedure can adjust the crimp and so compress at least a portion of the lumen.

In the depicted embodiments, the compression member 90 is formed from a biocompatible material. The ring can be formed of metallic material, such as stainless steel, titanium, or a suitable alloy. In another embodiment, the compression member can be formed of a biocompatible plastics materials such as ABS or polypropylene. The ring can be compressed in a manner such that its has a flat portion (as depicted in FIG. 15) or in another configuration, such as a zig-zag pattern as is depicted in FIG. 14. A compressing tool shaped to suit the dimensions of the crimp could be utilised in this regard.

As depicted in FIGS. 16a and 16b, the compression member can comprise a clip 100 which is mountable around the elongate member 30 and which can then be closed and latched as depicted in FIG. 16b. The dimensions of the clip relative to the elongate member are such that on closing and latching of the clip, at least a portion of the lumen 32 is compressed sufficiently to at least substantially seal the lumen. The clip 100 is capable of manipulation by the hands of the surgeon. A suitable tool for performing this function can, however, also be envisaged. Such a clip can be formed from a biocompatible plastics material, such as polypropylene.

While the region 102 of the lumen of lesser diameter depicted in FIG. 17 can be used in conjunction with the compression member 90 or the clip 100, the resiliently flexible elongate member can rely on this region to provide at least a partial barrier to transfer of pathogens through the lumen.

In the embodiment depicted in FIG. 17, the region 102 is relatively closer to the orifice 33 of the lumen 32 than at least one of said first lumen portions 102a.

The region 102 has a diameter that is about the same or less than the diameter of the stylet passing therethrough. In one embodiment, the diameter of the region 102 can be about 0.1 mm. The diameter of the portion 102a can be about 0.18 mm. In yet another embodiment, the region 102 can have a length of about 5 mm.

As depicted, the region 102 can be spaced from the orifice 33 of the lumen by at least one lumen portion 102b having a diameter greater than that of the region 102.

In this embodiment, the orifice 33 of the lumen can have a quantity of adhesive inserted therein that subsequently cures and seals the lumen. The adhesive can be a cyanoacrylate.

In this embodiment, at least the region 102 can also be coated with a material that swells on contact with at least certain fluids. For example, the region 102 of the lumen can have a coating of a hydrogel material that swells following contact with body fluids. The hydrogel can have a coating to allow control of the commencement time or rate of swelling of the hydrogel. The swelling of the hydrogel is such that the region 102 is sealed following withdrawal of the stylet.

The stylet used in conjunction with the invention can be formed of platinum but it will be appreciated that other biocompatible metals and plastics materials could be employed as a stylet in the present invention. The stylet used in conjunction with the elongate member is relatively stiffer than the silicone elongate member and is adapted to be positioned within the lumen thereof and so straighten the elongate member from its preferentially curved configuration to one that is more readily implantable in the cochlea of an implantee.

In each of the depicted embodiments, the elongate member 30 has a first straight or substantially straight configuration that allows the member 30 to be more readily inserted into an implantee's body, such as the cochlea. The member 30 is, however, pre-formed to preferentially adopt a second spirally-curved configuration when the stylet is not present so that the member 30 is more readily able to apply a preselected tissue stimulation with the electrodes.

The elongate member 30 is preformed from a suitable biocompatible material. In one embodiment, the material can be a silicone, such as Silastic MDX 4-4210. In another embodiment, the elongate member can be formed from a polyurethane or similar material.

Once implanted, the electrodes of the member 30 can receive stimulation signals from a stimulator device. The stimulator device is electrically connected to the elongate member by way of the electrical lead 21.

In one embodiment, the lead can extend from the elongate member to a stimulator device or at least the housing thereof. In one embodiment, the lead is continuous with no electrical connectors, at least external the housing of the stimulator device, required to connect the wires extending from the electrodes to the stimulator device.

The stimulator device is positioned within a housing that is implantable within the implantee. The housing for the stimulator device is implantable within the bony well in the bone behind the ear posterior to the mastoid.

When implantable, the housing contains, in addition to the stimulator device, a receiver device. The receiver device is able to receive signals from a controller. The controller is, in use, mounted external to the body of the implantee such that the signals are transmitted transcutaneously through the implantee.

Signals can travel from the controller to the receiver device and vice versa. The receiver device can include a receiver coil that is able to receive radio frequency (RF) signals from a corresponding transmitter coil worn externally of the body. The radio frequency signals can comprise frequency modulated (FM) signals. While described as a receiver coil, the receiver coil can transmit signals to the transmitter coil which receives the signals.

The transmitter coil is held in position adjacent the implanted location of the receiver coil by way of respective attractive magnets mounted centrally in, or at some other position relative to, the coils.

The external controller can comprise a speech processor that is able to receive signals output by a microphone. During use, the microphone is worn on the pinna of the implantee, however, other suitable locations can be envisaged, such as a lapel of the implantee's clothing. The speech processor encodes the sound detected by the microphone into a sequence of electrical stimuli following given algorithms, such as algorithms already developed for cochlear implant systems. The encoded sequence is transferred to the implanted receiver/stimulator device using the transmitter and receiver coils. The implanted receiver/stimulator device demodulates the FM signals and allocates the electrical pulses to the appropriate attached electrode by an algorithm which is consistent with the chosen speech coding strategy.

The external controller further comprises a power supply. The power supply can comprise one or more rechargeable batteries. The transmitter and receiver coils are used to provide power via transcutaneous induction to the implanted receiver/stimulator device and the electrode array.

While the implant system can rely on external componentry, in another embodiment, the controller, including the microphone, speech processor and power supply can also be implantable. In this embodiment, the controller can be contained within a hermetically sealed housing or the housing used for the stimulator device.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An implantable tissue-stimulating device comprising:
a resiliently flexible elongate member having a proximal end, a distal end, and having at least one electrode mounted thereon;
a lumen extending through at least a portion of said elongate member from an orifice positioned at or relatively closer to said proximal end than said distal end, said lumen being able to receive a stiffening element through said orifice; and
a seal that is pierceable by said stiffening element, said seal substantially sealing said lumen following removal of said stiffening element therefrom.

2. The implantable tissue-stimulating device of claim 1, wherein
said stiffening element extending through at least a portion of said lumen and out through said orifice;

3. The implantable tissue-stimulating device of claim 1 wherein a slit is formed in said seal.

4. The implantable tissue-stimulating device of claim 1 wherein said seal is formed of a silicone polymer.

5. A method of manufacturing an implantable tissue-stimulating device, the device comprising a resiliently flexible elongate member having a proximal end, a distal end and at least one electrode mounted thereon, and a lumen extending through at least a portion of the elongate member from an orifice positioned at or relatively closer to the proximal end than the distal end, the method comprising the steps of:
(i) sealing the lumen of the elongate member with a pierceable seal; and
(ii) piercing the seal with a tip of a stiffening element and sliding the stiffening element relatively into the lumen of the elongate member.

6. The method of manufacturing an implantable tissue-stimulating device if claim 5, futher comprising the step of:
positioning a stiffening element within a lumen of an elongate member, the stiffening element extending from within the lumen back out through an orifice of the lumen.

7. A method of placing an implantable tissue-stimulating device in a body of an implantee, the device comprising a resiliently flexible elongate member having a proximal end, a distal end and at least one electrode mounted thereon, a lumen extending through at least a portion of the elongate member from an orifice positioned at or relatively closer to the proximal end than the distal end, and a seal that at least substantially seals the lumen, the method comprising the steps of:
(i) inserting the elongate member into a desired location in the body of the implantee;
(ii) during and/or after insertion of the elongate member, at least partially relatively withdrawing the stiffening element from the lumen through the seal; and
(iii) allowing the seal to at least substantially seal the lumen of the elongate member.

8. The implantable tissue-stimulating device of claim 1, further comprising:
a plug member that is positionable within and seals said orifice of said lumen following withdrawal of said stiffening element therefrom.

9. (canceled)

10. The implantable tissue-stimulating device of claim 8 wherein said elongate member is resiliently flexible and has a second proximal end, a second distal end, and at least one electrode mounted thereon, said lumen extending through at least a portion of said elongate member from said orifice that is positioned at or relatively closer to said second proximal end than said second distal end.

11. The method of placing an implantable tissue-stimulating device in the body of an implantee of claim 7, further comprising the steps of:
during and/or after insertion of the elongate member, relatively withdrawing a stiffening element from the lumen through the orifice in the member; and
inserting a plug into the orifice to at least substantially seal the lumen of the elongate member.

12. An implantable tissue-stimulating device comprising:
a resiliently flexible elongate member having a proximal end, a distal end, and having at least one electrode mounted thereon;
a lumen extending through at least a portion of said elongate member from an orifice positioned at or relatively closer to said proximal end than said distal end;
a stiffening element positioned at least partially within said lumen and extending out of said lumen through said orifice; and
a sealing member mountable to said stiffening element;
wherein said stiffening element is movable relative to said orifice of said lumen between a first position in which said sealing member mountable thereon does not seal said lumen and a second position in which said sealing member substantially seals said lumen.

13. The implantable tissue-stimulating device of claim 12 wherein said sealing member comprises a sealing portion of a resiliently flexible material mounted to said stiffening element.

14. The implantable tissue-stimulating device of claim 12 wherein said sealing member has a shape that substantially matches the shape of a narrowing of the lumen at or adjacent said orifice thereof.

15. The implantable tissue-stimulating device of claim 12 wherein said sealing member comprises a substantially spherical shape or spherical member mounted at or relatively near said distal end of said stiffening member.

16. The implantable tissue-stimulating device of claim 15 wherein a portion of said lumen adjacent said orifice thereof has a spherical region to receive said spherical member when said spherical member is in a second position.

17. The implantable tissue-stimulating device of claim 16 wherein said spherical region has a diameter less than that of said spherical member.

18. (canceled)

19. The method of placing an implantable tissue-stimulating device in the body of an implantee of claim, the method further comprising the steps of:
bringing a sealing member that is mountable to the stiffening element into a position in which the sealing member at least substantially seals the lumen.

20. The implantable tissue-stimulating device of claim 12, further comprising:
a compression member mountable around at least a portion of said elongate member;
wherein said compression member is adjustable between a first configuration in which said compression member does not compress a portion of said lumen and a second configuration in which said compression member does compress at least a portion of said lumen.

21. The implantable tissue-stimulating device of claim 20 wherein said lumen is able to receive said stiffening element through said orifice thereof.

22. The implantable tissue-stimulating device of claim 20 wherein a position of said compression member around said elongate member is adjustable.

23. The implantable tissue-stimulating device of claim 22 wherein said position of said compression member is adjustable once from a first configuration to a second configuration.

24. The implantable tissue-stimulating device of claim 20 wherein said compression member is a clip that is mountable around said elongate member, wherein on closing and latching of said clip, at least a portion of said lumen is compressed sufficiently to at least substantially seal said lumen.

25. The implantable tissue-stimulating device comprising of claim 1,
wherein said lumen has first portion having a first diameter and at least one second portion having a second diameter less than that of said first diameter; and
wherein said second portion is relatively closer to said orifice of said lumen than said first portions.

26. The implantable tissue-stimulating device of claim 25 wherein said second portion is spaced from said orifice of said lumen by at least one first portion.

27. The implantable tissue-stimulating device of claim 25 wherein said second portion is compressible by a compression member that is mountable around said elongate member, wherein said compression member is adjustable between a first configuration in which said compression member does not compress said second portion of said lumen and a second configuration in which said compression member does compress said second portion of said lumen.

28. The implantable tissue-stimulating device of claim 25 wherein a quantity of adhesive is insertable through said orifice of said lumen, said adhesive sealing said lumen upon curing.

29. The implantable tissue-stimulating device of claim 25 wherein at least said second portion of said lumen is coated with a material that swells on contact with fluids.

30. The implantable tissue-stimulating device of claim 1
wherein at least a portion of said lumen is coated with a layer of material that swells following exposure to bodily fluids.

31. The implantable tissue-stimulating device of claim 1,
wherein said lumen in a region adjacent said orifice decreases in diameter away from said orifice into said elongate member for a length.

32. The implantable tissue-stimulating device of claim 31 wherein said region of said lumen is frusto-conical in form.

33. The implantable tissue-stimulating device of claim 31 wherein said region is packable with fibrous tissue following withdrawal of said stiffening element.
